(19) United States
(12) Patent Application Publication
(10) Pub. No.: US 2010/0016674 A1
(43) Pub. Date: Jan. 21, 2010

(54) VAGINAL SPECULUM

(76) Inventor: Steven C. Mills, Wheeling, WV (US)

Correspondence Address: ADAMS INTELLECTUAL PROPERTY LAW, Suite 2350 Charlotte Plaza, 201 South College Street, CHARLOTTE, NC, 28244, US

(21) Appl. No.: 12/356,233

(22) Filed: Jan. 20, 2009

Related U.S. Application Data
(60) Provisional application No. 61/081,239, filed on Jul. 16, 2008.

(51) Int. Cl. A61B 1/32; B65D 69/00

(52) U.S. Cl. 600/207; 206/569

(57) ABSTRACT

An inflatable vaginal speculum that includes diametrically and longitudinally expandable sidewalls with openings allowing observation of vaginal walls along the length of the speculum and a central opening allowing viewing down the length of the speculum to the cervix. The speculum may be positioned within an envelope together with a sterile saline solution. An array of the envelopes can be stored in a storage box together with bulb-type pump such as used with a blood pressure cuff. The envelope is opened by tearing down one side so that the speculum can be removed.

### CROSS-REFERENCE TO RELATED APPLICATION

This application claims priority to U.S. Provisional Patent Application No. 61/081,239 filed Jul. 16, 2008 by Mills and entitled “VAGINAL SPECULUM,” the contents of which are incorporated by reference herein.

### TECHNICAL FIELD AND BACKGROUND OF THE INVENTION

This invention relates to a vaginal speculum, and particularly to a single-use, inflatable speculum that solves several problems inherent in currently-used specula. Typical vaginal specula are rigid plastic or metal instruments used to spread the walls of the vagina so that the examiner can inspect the cervix, the tapered bottom part of the uterus, with an opening that joins the uterus to the vagina. With the speculum inside the vaginal cavity, the examiner slowly thrusts the walls of the cavity apart by spreading the ‘spoons’ of the specula away from each other to inspect the vagina and cervix for any discharge or other related signs of illness. The speculum is also used spread the vagina so that cells from the cervix can be obtained for medical testing, such as the common test known as a Pap smear.

Typical specula do not provide a full 360 degree visual field. Several penetrations may therefore be needed to fully inspect the entire vaginal area and collect the necessary cells. Such specula can also cause the vaginal tissues to be pinched or bruised. In cases presenting atrophic vaginal tissue or in obese patients, a dramatic decrease in canal visibility is often the case. In patients with anteverted or retroverted uteri, the speculum is used as a search implement, and is manipulated within the vagina as needed to locate the cervix. As a patient gets more nervous and tense she may involuntarily squeeze her legs closed, causing pain due to the hard, unyielding edge of the speculum.

Metal specula are inherently cold and therefore uncomfortable to the patient unless carefully warmed. They must be cleaned and autoclaved after each use, requiring bactericidal solutions and autoclaving equipment. Plastic specula have sharp edges and a tendency to collapse during the examination and cell collection process. With both metal and plastic specula, a prior unpleasant examination experience can result in the patient delaying or avoiding regular examinations.

Therefore, there is a need for a speculum that has no rigid edges, is transparent, uses air to enlarge the speculum and spread the vaginal walls, and is disposable. The present invention fulfills these needs and provides a vaginal speculum that enables a physician to conduct a more thorough and comfortable vaginal examination.

### BRIEF SUMMARY OF THE INVENTION

Therefore it is an object of the present invention to provide an inflatable vaginal speculum that includes diametrically and longitudinally expandable sidewalls with openings allowing observation of vaginal walls along the length of the speculum and a central opening allowing viewing down the length of the speculum to the cervix. In at least one embodiment, the diametrically expandable sidewalls comprise air columns that encircle the central opening upon inflation. The air columns in at least one example are spaced and are progressively longer to provide a shape that enlarges along its length. The longitudinally expandable sidewalls may be in communication with the diametrically expandable sidewalls to permit inflation through an inflation tube. A handle for remaining exterior to the vagina, and a flap for covering a finger to permit the speculum to be inserted may each be included. The sidewalls may be formed of a flexible, expandable non-latex plastic material such as polyethylene. The speculum may be packaged in a pouch containing a sterile saline solution.

It is another object of the present invention to provide a kit of inflatable and disposable specula comprising multiple inflatable vaginal specula each including diametrically and longitudinally expandable sidewalls with openings allowing observation of vaginal walls along the length of the speculum and a central opening allowing viewing down the length of the speculum to the cervix. An inflation device for inflating the specula may be included in the kit. Each speculum may be packaged in a respective pouch in a sterile condition. Each pouch may contain a sterile saline solution.

### BRIEF DESCRIPTION OF THE DRAWINGS

Some of the objects of the invention have been set forth above. Other objects and advantages of the invention will appear as the description of the invention proceeds when taken in conjunction with the attached drawings, which illustrate embodiments of the invention and which are incorporated by reference herein:

FIG. 1 is a view of a dispensing box containing an array of packaged specula according to an embodiment of the invention;

FIG. 2 is a view of a single speculum in its sterile packaging;

FIG. 3 is a view of a speculum gathered onto the index and middle finger in preparation for insertion;

FIG. 4 is a fragmentary view of the speculum, showing the fingers within a forward flap; and

FIG. 5 is a modified cross-sectional environmental view of the inflated speculum.

### DETAILED DESCRIPTION OF THE PREFERRED EMBODIMENT AND BEST MODE

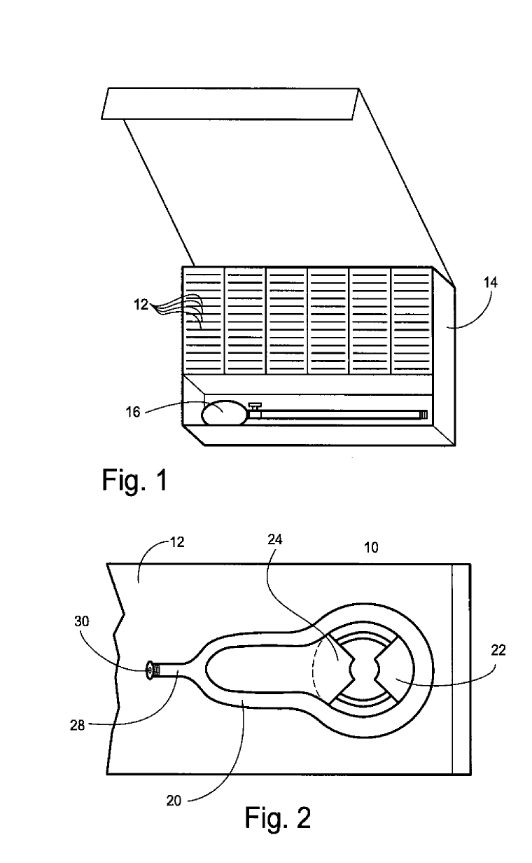

Referring now to FIG. 1, speculum 10 according to one embodiment of the invention is individually packaged in, for example, a plastic or laminated plastic/foil envelope 12 similar to packages commonly used to package condoms. Preferably, the speculum 10 is sufficiently expandable that one size can be used.

Each speculum 10 is contained within the envelope 12 together with a sterile saline solution. An array of the envelopes 12 can be stored in a storage box 14 together with bulb-type pump 16 such as used with a blood pressure cuff. As shown in FIG. 2, the envelope 12 is opened by tearing down one side so that the speculum 10 can be removed. The speculum 10 is formed of a flexible, expandable non-latex plastic material, for example, polyethylene. Such materials naturally tend towards ambient temperature and do not require warming. The material is also naturally slippery so a lubricant is not required. The material is preferably transparent.

The speculum 10 is stored in a deflated, collapsed condition, and includes a handle 20, and expandable sidewalls 22, 24. A plastic insert 26 in the handle 20 may be provided to provide rigidity to that part of the speculum 10 that remains at all times exterior to the vagina. An inflation tube 28 with a threaded nipple 30 permits air to be introduced into and evacuated from the speculum 10, as desired.

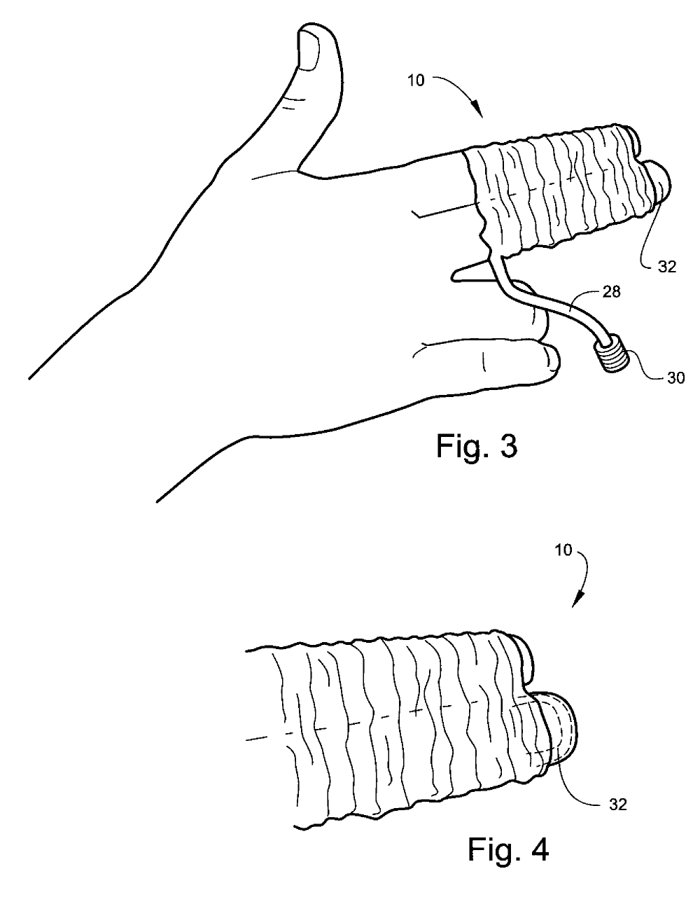

As is shown in FIG. 3, the speculum 10 is placed on the index and middle fingers in a collapsed, longitudinally-compressed condition. As is best shown in FIG. 4, a small flap 32 on the anterior end of the speculum 10 covers at least one finger and permits the fingers to extend into the vagina without the speculum 10 being forced backwards onto the fingers as insertion takes place.

The fingers with the speculum 10 in place are inserted into the vagina to essentially their full length. The pump 16 may be attached either before insertion or after insertion has taken place. In either case, the pump 16 is used in the normal manner. Air is introduced into the speculum 10, causing the speculum 10 to inflate. Inflation causes both an increase in the diameter and length of the speculum 10 that can be precisely controlled by the physician. Further manipulation of the speculum 10 can be accomplished as inflation takes place to, for example, more exactly orient the entrance of the cervix in alignment with the open central area of the speculum 10.

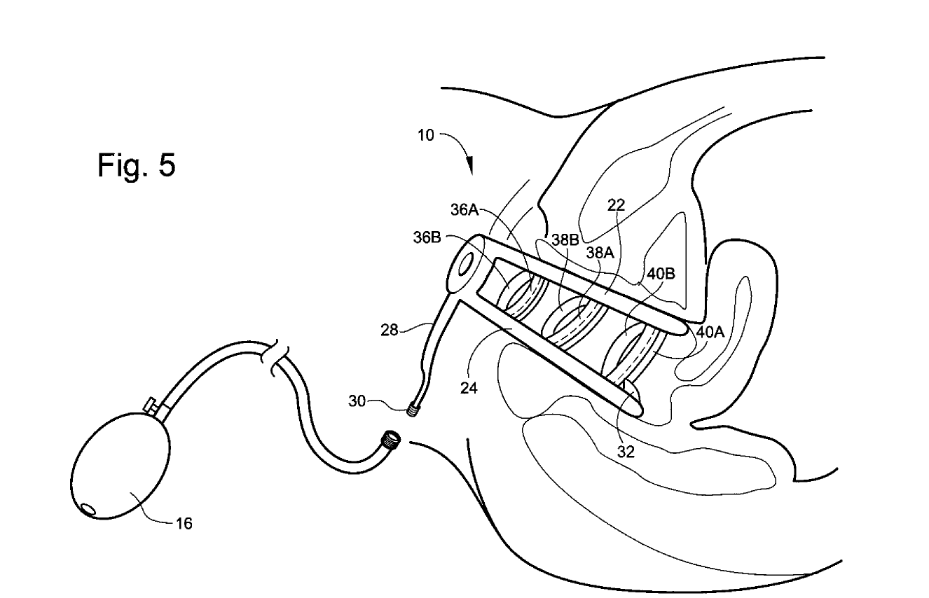

Referring now to FIG. 5, the speculum 10 in its inflated position within the vagina is shown. As can now be seen, the speculum 10 includes three pairs of air columns 36A, 36B, 38A, 38B and 40A, 40B. Each of these columns communicates with the inflation tube 28 and the side walls 22, 24, enabling inflation as described above. The air columns 36A, 36B, 38A, 38B and 40A, 40B are progressively longer, providing a shape that gradually enlarges along the length of the vagina to provide suitable expansion and a clear view of the cervix. In addition, the air columns 36A, 36B, 38A, 38B and 40A, 40B provide sufficient space between them such that the physician has an essentially 360 degree view inside the vagina with minimal reorientation of the speculum 10.

When performing an examination that includes taking a Pap smear, the physician only enters the vagina once. A bimanual exam is performed and the cervix is located. When the physician is ready to collect samples from the exo- and endo-cervix the speculum 10 is gently inflated. When the desired view is achieved, the fingers are removed and a spatula is inserted, as shown in FIG. 5. Because there is only one entry into the vagina, the examination is completed in half the time. If the patient gets nervous or embarrassed and squeezes her legs, she does so against air and not metal.

An inflatable speculum is described above. Various details of the invention may be changed without departing from its scope. Furthermore, the foregoing description of the preferred embodiment of the invention and the best mode for practicing the invention are provided for the purpose of illustration only and not for the purpose of limitation—the invention being defined by the claims.

What is claimed is:

1. An inflatable vaginal speculum that includes diametrically and longitudinally expandable sidewalls for insertion into a vagina with openings allowing observation of vaginal walls along the length of the speculum and a central opening allowing viewing down the length of the speculum to the cervix.

2. A speculum according to claim 1, wherein the diametrically expandable sidewalls comprise air columns that encircle the central opening upon inflation.

3. A speculum according to claim 2, wherein the air columns are spaced and are progressively longer to provide a shape that enlarges along its length.

4. A speculum according to claim 2, wherein the air columns are spaced to define the openings.

5. A speculum according to claim 1 wherein the longitudinally expandable sidewalls are in communication with the diametrically expandable sidewalls to permit inflation through an inflation tube.

6. A speculum according to claim 1, further comprising a handle for remaining exterior to the vagina during penetration by the sidewalls.

7. A speculum according to claim 1, further comprising a flap for receiving a finger of the user against which the finger may be urged to provide forward motion of the sidewalls into the vagina.

8. A speculum according to claim 1, wherein the sidewalls are formed of a flexible, expandable non-latex plastic material.

9. A speculum according to claim 8, wherein the material comprises polyethylene.

10. A speculum according to claim 1, further including a pouch in which the speculum is packaged in a sterile condition.

11. A speculum according to claim 1, further including a sterile saline solution within the pouch.

12. A kit of inflatable and disposable specula comprising multiple inflatable vaginal specula each packaged in a respective pouch and including diametrically and longitudinally expandable sidewalls with openings allowing observation of vaginal walls along the length of the speculum and a central opening allowing viewing down the length of the speculum to the cervix.

13. A kit according to claim 12, further comprising an inflation device for inflating the specula.

14. A kit according to claim 12, wherein each speculum is packaged in the respective pouch in a sterile condition.

15. A kit according to claim 14, wherein each pouch also contains a sterile saline solution.

16. A kit according to claim 12, further comprising a storage box containing the pouches within which the respective specula are packaged.
